(19) United States
(12) Patent Application Publication
(10) Pub. No.: US 2024/0041683 A1
(43) Pub. Date: Feb. 8, 2024

(54) METHOD FOR IMPROVING EYESIGHT

(71) Applicant: Leonard Gordon Hanson, Surrey (CA)

(72) Inventor: Leonard Gordon Hanson, Surrey (CA)

(21) Appl. No.: 17/816,776

(22) Filed: Aug. 2, 2022

(51) Int. Cl. A61G 10/02; A61H 5/00

(52) U.S. Cl. CPC A61G 10/026; A61H 5/00

(57) ABSTRACT

An eyesight improvement method operable to reduce or eliminate the effects of eye disease such as but not limited to presbyopia wherein the present invention deploys utilization of a hyperbaric chamber. The present invention includes utilization of a hyperbaric chamber wherein the participant in the method of the present invention will engage in a consecutive quantity of treatment sessions to be executed during a prescribed treatment term. The treatment sessions can range from fifteen to one hundred and fifty minutes with a preferred embodiment time of ninety minutes. The pressure within the hyperbaric chamber is set at a range of one and a half to two and a half atmospheric pressure with a preferred pressure at two atmospheric pressure. The preferred embodiment includes a treatment term of three months with a range being between one and five months.

### FIELD OF THE INVENTION

The present invention relates generally to medical treatments, more specifically but not by way of limitation, a hyperbaric treatment method that is operable to treat presbyopia resulting in the elimination of the effects thereof for older individuals.

### BACKGROUND

Presbyopia is physiological insufficiency of accommodation associated with the aging of the eye that results in an inability to focus clearly on objects that are close to the individual. This condition is also known as age-related farsightedness and it affects most adults over the age of forty. A common sign of presbyopia is difficulty reading small print which results in having to hold reading material farther away. This condition can further cause other symptoms such as but not limited to eyestrain and headaches. Presbyopia is a typical part of the aging process. It occurs due to age related changes in the lens of the eye, specifically the lens with age has decreased elasticity and increased hardness. Additionally, age results in decreased strength of the ciliary muscle which is the muscle responsible for controlling the movement of the lens of the eye. Presbyopia is often corrected using various types of eyeglasses or contact lens. On some occasions, procedures such as LASH will be utilized. People over forty are at risk for developing presbyopia and all people become affected to some degree.

Hyperbaric medicine, also known as hyperbaric oxygen therapy is the medical use of oxygen at a higher than normal atmospheric pressure. Hyperbaric oxygen therapy refers to intermittent treatment of the entire body with 100-percent oxygen at greater than normal atmospheric pressure. The earth's atmosphere normally exerts about 15 pounds per square inch of pressure at sea level. That pressure is defined as one atmosphere. In the ambient atmosphere we normally breathe approximately twenty percent oxygen and eighty percent nitrogen. While undergoing hyperbaric oxygen therapy, pressure is increased up to two times normal atmospheric pressure with the patient breathing one hundred percent oxygen. This increased pressure, combined with the increase in oxygen dissolves oxygen in the blood plasma and in all body cells, tissues and fluids at up to ten times normal concentration. The benefits of this therapy can include but are not limited to: increased oxygen concentration in all body tissues, stimulation of growth of new blood vessels to locations with reduced circulation which improves blood flow to areas with arterial blockage, improving blood flow to compromised organs and can aid in the treatment of infection by enhancing white blood cell action. While benefits of hyperbaric therapy are known, no known treatment method exists for eliminating presbyopia.

It is intended within the scope of the present invention to provide an eyesight improvement method utilizing hyperbaric treatments wherein the present invention is operable to eliminate the effects of presbyopia for older adults.

### SUMMARY OF THE INVENTION

It is the object of the present invention to provide a treatment method for treating eyesight maladies such as but not limited to presbyopia wherein the present invention includes the utilization of a hyperbaric chamber.

Another object of the present invention is to provide an eyesight improvement method that is operable to diminish or eliminate the effects of presbyopia for older adults wherein the present invention includes a term of hyperbaric chamber that is at least three months.

A further object of the present invention is to provide a treatment method for treating eyesight maladies such as but not limited to presbyopia wherein the method of the present invention includes utilization of a pressure range of one and a half to two atmospheric pressures during treatment session that occur during the at least three month term.

Still another object of the present invention is to provide an eyesight improvement method that is operable to diminish or eliminate the effects of presbyopia for older adults wherein the preferred embodiment includes at least five sessions per week during the at least three month term.

An additional object of the present invention is to provide a treatment method for treating eyesight maladies such as but not limited to presbyopia wherein the five session per week during the at least three month term are preferred to be within a range of fifteen to one hundred and fifty minutes each in length.

Yet a further object of the present invention is to provide an eyesight improvement method that is operable to diminish or eliminate the effects of presbyopia for older adults wherein the treatment term within the scope of the preferred embodiment is one to five months in length.

Another object of the present invention is to provide a treatment method for treating eyesight maladies such as but not limited to presbyopia wherein the present invention can employ either a monoplace chamber or multiplace chamber hyperbaric chamber.

To the accomplishment of the above and related objects the present invention may be embodied in the form illustrated in the accompanying drawings. Attention is called to the fact that the drawings are illustrative only. Variations are contemplated as being a part of the present invention, limited only by the scope of the claims.

### BRIEF DESCRIPTION OF THE DRAWINGS

A more complete understanding of the present invention may be had by reference to the following Detailed Description and appended claims when taken in conjunction with the accompanying Drawings wherein:

FIG. 1 is an outline diagram of the method of the present invention.

### DETAILED DESCRIPTION

Referring now to the drawings submitted herewith, wherein various elements depicted therein are not necessarily drawn to scale and wherein through the views and figures like elements are referenced with identical reference numerals, there is illustrated an eyesight improvement method 100 constructed according to the principles of the present invention.

An embodiment of the present invention is discussed herein with reference to the figures submitted herewith. Those skilled in the art will understand that the detailed description herein with respect to these figures is for explanatory purposes and that it is contemplated within the scope of the present invention that alternative embodiments are plausible. By way of example but not by way of limitation, those having skill in the art in light of the present teachings of the present invention will recognize a plurality of alternate and suitable approaches dependent upon the needs of the particular application to implement the functionality of any given detail described herein, beyond that of the particular implementation choices in the embodiment described herein. Various modifications and embodiments are within the scope of the present invention.

It is to be further understood that the present invention is not limited to the particular methodology, materials, uses and applications described herein, as these may vary. Furthermore, it is also to be understood that the terminology used herein is used for the purpose of describing particular embodiments only, and is not intended to limit the scope of the present invention. It must be noted that as used herein and in the claims, the singular forms “a”, “an” and “the” include the plural reference unless the context clearly dictates otherwise. Thus, for example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

References to “one embodiment”, “an embodiment”, “exemplary embodiments”, and the like may indicate that the embodiment(s) of the invention so described may include a particular feature, structure or characteristic, but not every embodiment necessarily includes the particular feature, structure or characteristic.

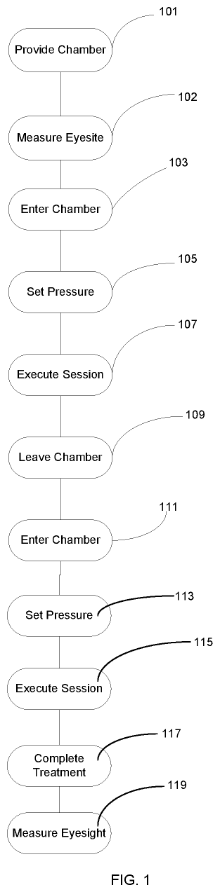

Now referring to the drawing submitted herewith, the eyesight improvement method 100 is outlined in its preferred embodiment in FIG. 1 submitted as a part hereof. It is contemplated within the scope of the present invention that the variations could occur wherein these variations could be but are not limited to adjustments of atmospheric pressure, treatment term and session length adjustments. Variations are based upon the individual, the health of the individual and the desired outcome by the individual.

The eyesight improvement method 100 initiates with step 101 wherein an operator of the eyesight improvement method 100 will provide a hyperbaric chamber. The hyperbaric chamber provided is a conventional hyperbaric chamber providing a one hundred percent oxygen air supply to the individual disposed therein and capable of further increasing the atmospheric pressure within the hyperbaric chamber. It is contemplated within the scope of the present invention that the eyesight improvement method 100 can be deployed utilizing a monoplace or a multiplace hyperbaric chamber. In step 102, an operator of the eyesight improvement method 100 will measure and record the eyesight of the individual whom will engage in the method of the present invention. Step 103, the individual will enter the interior of the provided hyperbaric chamber. In step 105, the pressure for the interior of the hyperbaric chamber is set. It should be understood within the scope of the present invention that the pressure within the hyperbaric chamber is set within a range of one and a half atmospheric pressure to two and a half atmospheric pressure with the preferred atmospheric pressure for each treatment session being two atmospheric pressure.

In step 107, the operator will initiate and execute the treatment session for the individual disposed within the interior of the hyperbaric chamber. While alternate lengths of time can be utilized for the treatment sessions, it is desired within the scope of the present invention that the treatment session be provided within a range of fifteen to one hundred and fifty minutes with a preferred length of time being ninety minutes. Step 109, ensuing completion of the treatment session, the individual will exit the hyperbaric chamber. In step 111, on a consecutive day, the individual will return to the provider of the hyperbaric chamber to initiate a subsequent treatment session and enter the hyperbaric chamber. Step 113, the operator of the eyesight improvement method 100 will set the pressure of the hyperbaric chamber to a pressure previously discussed herein. In step 115, the individual will complete the treatment session wherein the length of time for the treatment session is delivered as discussed herein. In step 117, the individual will engage in consecutive treatment sessions in order to fulfill the requirement for the treatment term. While alternate treatment terms can be employed, in the preferred embodiment of the present invention the treatment term is three month with five consecutive treatment session during each week of the treatment term. It is contemplated within the scope of the present invention that the treatment term could range between one month and five months with each term having five consecutive treatment session each week during the prescribed term. Step 119, ensuing completion of the treatment term the operator of the eyesight improvement method 100 will measure the eyesight of the individual.

In the preceding detailed description, reference has been made to the accompanying drawings that form a part hereof, and in which are shown by way of illustration specific embodiments in which the invention may be practiced. These embodiments, and certain variants thereof, have been described in sufficient detail to enable those skilled in the art to practice the invention. It is to be understood that other suitable embodiments may be utilized and that logical changes may be made without departing from the spirit or scope of the invention. The description may omit certain information known to those skilled in the art. The preceding description is, therefore, not intended to be limited to the specific forms set forth herein, but on the contrary, it is intended to cover such alternatives, modifications, and equivalents, as can be reasonably included within the spirit and scope of the invention.

What is claimed is:

1. A method for treating eyesight deficiencies wherein the method comprises the steps of:
providing a hyperbaric chamber, said hyperbaric chamber configured to provide an increased atmospheric pressure in an interior volume thereof and further provide a one hundred percent oxygen supply;
measuring a first eyesight reading of a participant in the method;
prescribing a treatment term, wherein the treatment term of utilization of the hyperbaric chamber is determined for the participant;
determining a length of time for treatment sessions, wherein a quantity of time for treatment sessions for the participant is calculated;
executing the treatment sessions, wherein the participant will engage in the treatment session wherein the treatment sessions consist of consecutive sessions during the treatment term;
completing the treatment term, wherein the participant completes all of the treatment sessions within the treatment term; and
measuring a second eyesight reading of the participant, wherein the second eyesight reading of the participant is compared to the first eyesight reading.

2. The method for treating eyesight deficiencies as recited in claim 1, wherein a preferred time for a treatment session is ninety minutes.

3. The method for treating eyesight deficiencies as recited in claim 2, wherein a preferred range of time for the treatment sessions is fifteen to one hundred and fifty minutes.

4. The method for treating eyesight deficiencies as recited in claim 3, and further including a step of setting a pressure for the hyperbaric chamber, wherein the pressure is set within a range of one and a half to two and a half atmospheric pressures.

5. The method for treating eyesight deficiencies as recited in claim 4, wherein a preferred embodiment provides a setting of two atmospheric pressures for the treatment session.

6. The method for treating eyesight deficiencies as recited in claim 5, wherein the treatment term ranges between one to five months.

7. The method for treating eyesight deficiencies as recited in claim 6, wherein a preferred embodiment of the treatment term is three months.

8. The method for treating eyesight deficiencies as recited in claim 7, wherein the consecutive sessions of treatment session include five consecutive treatment sessions each week of the treatment term.
